(19) United States
(12) Patent Application Publication
(10) Pub. No.: US 2026/0137842 A1
(43) Pub. Date: May 21, 2026

(54) DRUG COATED DEVICES AND METHODS FOR THE TREATMENT OF EYE DISEASE

(71) Applicant: J.D. Franco & Co., LLC, Plano, TX (US)

(72) Inventors: Edward PARSONAGE, St. Paul, MN (US); Stephen MICHAEL, Maple Grove, MN (US); Zachary TEGELS, Minneapolis, MN (US); Michael CALHOUN, Lighthouse Point, FL (US)

(73) Assignee: J.D. Franco & Co., LLC, Plano, TX (US)

(21) Appl. No.: 18/857,905

(22) PCT Filed: May 10, 2023
(86) PCT No.: PCT/US2023/066830

§ 371 Date: Oct. 18, 2024

Related U.S. Application Data
(60) Provisional application No. 63/364,505, filed on May 11, 2022.

(51) Int. Cl. A61L 29/16; A61L 29/08; A61L 31/08; A61L 31/16

(52) U.S. Cl. CPC A61L 29/16; A61L 29/08; A61L 31/08; A61L 31/16

(57) ABSTRACT

An intravascular device including a transferable drug coating adhered to an exposed outer surface on the device, the drug coating comprising an anti-proliferative active ingredient and a xanthophyll excipient. The xanthophyll may be selected from the group consisting of lutein, astaxanthin, zeaxanthin, meso zeaxanthin, cryptotaxanthin, tunaxanthin, salmoxanthin, and parasiloxanthin, and mixtures thereof. The anti-proliferative active ingredient may be selected from the group consisting of sirolimus, everolimus, zotarolimus, bioliimus, tacrolimus, pimecrolimus, paclitaxel, and mixtures thereof. The device may be used in the treatment of eye disease.

### CROSS-REFERENCE TO RELATED APPLICATION

This application claims the benefit of priority of U.S. Provisional Patent Application No. 63/364,505, filed on May 11, 2022, which is incorporated by reference herein in its entirety.

### FIELD

The present disclosure relates to drug coatings applied to intravascular devices for the treatment of vascular stenosis.

### BACKGROUND

Drug coated balloons have been demonstrated to successfully treat atherosclerotic lesions in balloon angioplasty. (See, for example, Picard et. al. 2017). At present, the most successful treatment indications using this procedure have been for lower peripheral arteries such as superficial femoral and lower popliteal arteries. However, it is generally understood that significant loss of drug coating occurs during tracking of the catheter to the lesion site and subsequent deployment of the balloon. Furthermore, a significant portion of drug coating that is transferred to the lesion site during balloon expansion may be washed downstream of the lesion site after removal of the balloon. To compensate for this loss, the amount of drug loaded on the balloon is proportionally increased. Furthermore, an additional component is usually combined with the drug. This additional component is generally known as an excipient. The purpose of the excipient is to increase the pharmacokinetic tissue levels of drug after deployment via several mechanisms. First, the excipient can improve the toughness and adhesion of the drug coating to the balloon to minimize particulation during delivery and expansion of the balloon. Second, the excipient can affect the fractured drug coating particle size to further improve transfer of drug to the lesion site. Finally, the excipient can impact the rate of dissolution of drug lodged in the tissue, thus extending the time for achieving clinically relevant tissue levels. In practice, it is not possible to completely eliminate drug particulation and loss to regions downstream of the target lesion site. Therefore, it is important that, in addition to the drug, the excipient also has an appropriate safety dosage window for any exposed tissue and vessels within the distal vessel bed.

### References

Fabien Picard, Serge Doucet, Anita W. Asgara
Archives of Cardiovascular Diseases
Volume 110, Issue 4, April 2017, Pages 259-272.
A. V. Rao, L. G. Rao
Pharmacological Research 55 (2007) 207-216.
Paul S. Bernstein, Binxing Lia, Preejith P. Vachalia, Aruna Gorusupudia, Rajalekshmy Shyama, Bradley S. Henriksena, and John M. Nolan
Prog Retin Eye Res. 2016 January; 50:34-66.
U.S. Pat. No. 9,375,329B2; EP1362603B1; U.S. Pat. No. 8,241,249B2

### SUMMARY

Described herein is an intravascular carrier device including a transferable drug coating adhered to an exterior surface of the device. In general, the drug coating may contain both an anti-proliferative therapeutic agent and an excipient. In some embodiments, the therapeutic agent may be selected from the group: sirolimus, everolimus, zotarolimus, biolimus, tacrolimus and pimecrolimus, and paclitaxel. In some embodiments, the excipient may be a xanthophyll. In further embodiments, the xanthophyll excipient may be selected from the group: lutein, astaxanthin, zeaxanthin, meso zeaxanthin, cryptotaxanthin, tunaxanthin, salmoxanthin, and parasiloxanthin, to name a few.

In some embodiments, the drug coating may be adhered to a balloon surface of an intravascular balloon catheter, wherein the balloon is in an expanded configuration. In further embodiments, the drug coating may be adhered to the balloon surface wherein the balloon is in a folded configuration.

In another embodiment, a method for treating a stenotic arterial or venous stenosis or lesion may comprise the steps of: providing an intravascular carrier device such as a balloon catheter on which a transferable drug coating is adhered to an outer surface such as an inflatable balloon, wherein the drug coating comprises an anti-proliferative active ingredient and a xanthophyll excipient, expanding a portion of intravascular device such as inflating the drug-coated inflatable balloon within the lumen contacting the stenosis, and transferring the drug coating from the balloon surface to the tissue comprising the stenotic lesion.

The concepts described herein may be applied to diseases of the eye. Examples of eye diseases and conditions include, but are not limited to: Age-Related Macular Degeneration (AMD), both dry and wet conditions; neuronal cell death; Alzheimer's disease; dementia; glaucoma; diabetic macula edema; macular telangiectasia (e.g., type 1 or 2 macular telangiectasia); atrophic macular degeneration; chorioretin-apathy (e.g., central serous chorioretinopathy); retinal inflammatory vasculopathy; pathological retinal angiogen-esis; age-related maculopathy; retinoblastoma; Pseudox-anthoma elasticum; a vitreoretinal disease; choroidal sub-retinal neovascularization; central serous chorioretinopathy; ischemic retinopathy; hypertensive retinopathy or diabetic retinopathy (e.g., nonproliferative or proliferative diabetic retinopathy, such as macular edema or macular ischemia); retinopathy of prematurity (e.g., associated with abnormal growth of blood vessels in the vascular bed supporting the developing retina); venous occlusive disease (e.g., a retinal vein occlusion, branch retinal vein occlusion or central retinal vein occlusion); arterial occlusive disease (e.g., branch retinal artery occlusion (BRAO); central retinal artery occlusion or ocular ischemic syndrome); central serous chorioretinopathy (CSC); cystoid macular edema (CME) (e.g., affecting the central retina or macula, or after cataract surgery); retinal telangiectasia (e.g., characterized by dilation and tortuosity of retinal vessels and formation of multiple aneurysms, idiopathic JXT, Lebar's miliary aneu-rysms, or Coats'disease); arterial microaneurysm; retinal angiomatosis; radiation-induced retinopathy (RIRP); or rubeosis iridis (e.g., associated with the formation of neo-5 vascular glaucoma, diabetic retinopathy, central retinal vein occlusion, ocular ischemic syndrome, or chronic retinal detachment); distortions and/or blind spots (scotoma); changes in dark adaptation (diagnostic of rod cell health); changes in color interpretation (diagnostic of cone cell 10 health); decrease in visual acuity; and cataracts (e.g., age-related cataract).

The pathogenesis of some of these eye diseases may be similar if not the same as those seen in arterial diseases including: coronary artery disease, peripheral vascular disease, etc. However, the anatomy of the vasculature behind the eye is typically smaller, includes more branches, and includes more sharp angles in the blood flow pathway. Further, the vascular system supplying blood to the eye is closer to the brain; any uncaptured or non-rerouted debris may cause an immediate stroke.

### BRIEF DESCRIPTION OF THE DRAWINGS

FIG. 1 shows a balloon catheter with an inflatable balloon connected to both an inner and outer shaft, with the lumen between the inner and outer shaft defining an inflation lumen.

FIG. 2 a-h show various intravascular devices which may serve as a carrier for a drug, wherein a drug coating is applied to any or all of the surfaces of each respective device, such as an expandable portion of the device. This list of devices is not meant to be limiting. Any device used within the vascular system may be considered for application of the drug coating.

FIG. 3 a-b show an inflatable balloon having a drug coating adhered to the outer surface. The drug coating may be applied in both a folded or inflated configuration.

FIG. 4 shows the general chemical structure of a xanthophylls. In accordance with the present disclosure, a xanthophyll may be defined as any chemical compound wherein at least one carbon of pendant group c1-6, or pendant group c1′-c6′ is also chemically bonded to oxygen.

FIG. 5 a-c show the chemical structure of lutein, zeaxanthin, and meso-zeaxanthin, respectively.

FIG. 6 shows an image associated with Example 1 demonstrating the transfer of drug coating from an inflated balloon to an arterial surface.

FIG. 7 shows an image associated with Example 2 demonstrating the transfer of drug coating from an inflated balloon to an arterial surface.

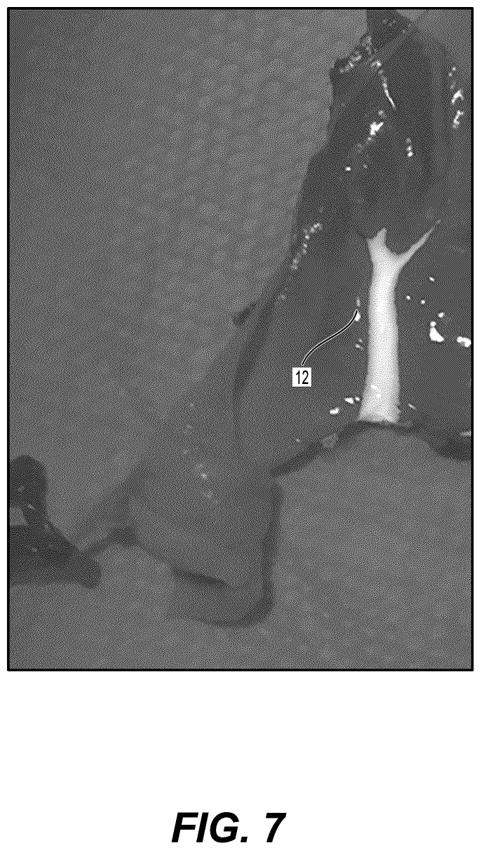

### DETAILED DESCRIPTION

### Balloon Catheter

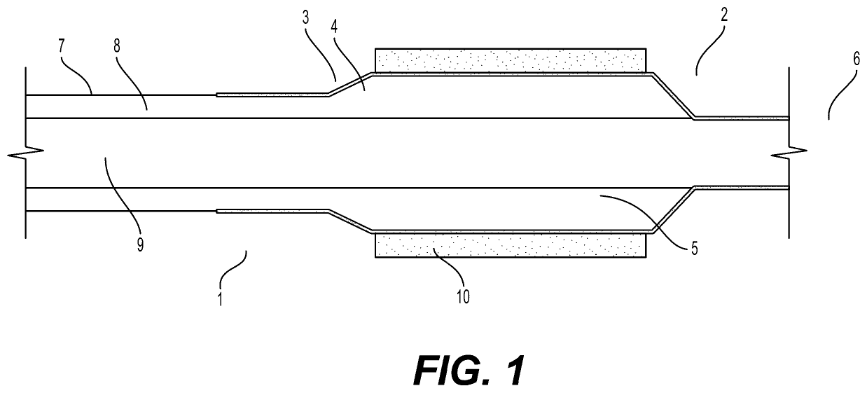

In accordance with the present disclosure, a balloon catheter is provided as shown in FIG. 1. The balloon catheter 1 has an inflatable balloon 2 with an inner surface 3 and outer surface 4. An inner shaft 5 is bonded to the inner surface 3 at the distal end of the balloon 6. An outer shaft 7 is bonded to the proximal end of the inflatable balloon 2 creating a lumen 8 used to inflate the balloon via fluid communication with the proximal end of the catheter. The inner lumen 9 of the inner shaft may be used to deliver the balloon catheter using a guidewire.

In further accordance with the present disclosure, a transferable drug coating 10 is adhered to the outer surface 4 of the balloon. The degree of adhesion between the drug coating and balloon is generally sufficient as to allow delivery of the balloon to the lesion site without significant loss of coating, while at the same time allowing transfer of a clinically efficacious amount of drug onto the vascular tissue upon inflation of the balloon.

Inflatable balloons for catheters are generally categorized as compliant or non-compliant. Examples of compliant balloons include those comprised of polyurethanes, polyether-amide thermoplastic elastomers and nylon 12, to name a few. Examples of non-compliant balloons include those comprised of polyethylene terephthalate and expanded polytetrafluoroethylene, as examples.

### Other Intravascular Device Carriers

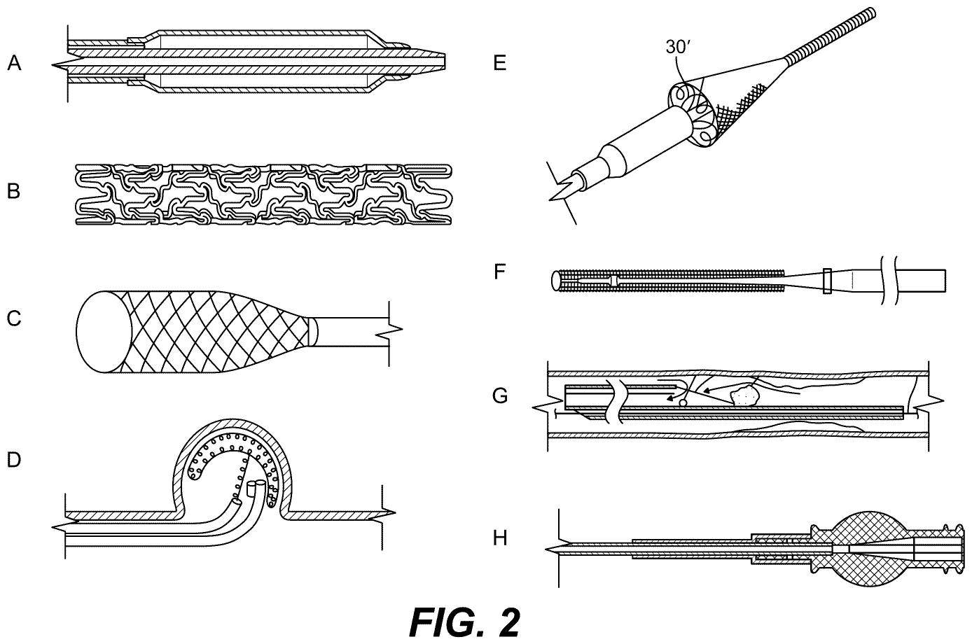

As an alternative to using a balloon catheter as a carrier for the drug coating, other devices may be used to deliver drugs to a desired location. As previously mentioned, FIG. 2A shows a balloon catheter which may be used as a carrier. In addition, other devices used during intra-vascular procedures may also be considered for use with drug coating. Examples include a stent as shown in FIG. 2B, a stent retriever as shown in FIG. 2C, an aneurysm coil as shown in FIG. 2D, a filter as shown in FIG. 2E, a guidewire as shown in FIG. 2F, an aspiration catheter as shown in FIG. 2G, and a microcatheter as shown in FIG. 2H.

### Coating Process

The drug coating 10 may be applied to an exposed surface of the intravascular device, particularly a portion that comes into contact with the stenosis, such as a balloon surface. The drug coating 10 may be applied by any process resulting in a clinically efficacious dosage applied to the surface. The coating may be applied, for example, by spray coating, electrospray, dip coating, roll coating, direct write coating such as aerosol jet, ink jet or micropen application, and powder coating, to name a few. The coating process may further include a volatile solvent which is subsequently removed from the coating. The coating process may further include a drying process such as elevated temperature drying and annealing within a convection oven.

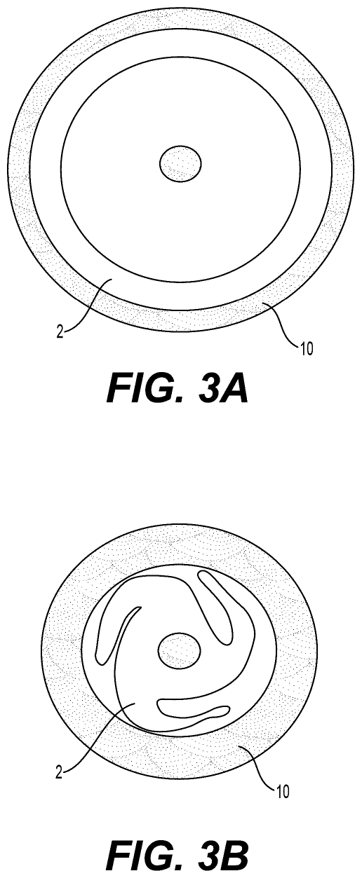

Inflatable balloons associated with catheter angioplasty are often delivered to the lesion site in a folded configuration. As shown in FIG. 3a, in further accordance with the present disclosure, the drug coating 10 may be applied to the balloon 2 in the expanded configuration. Alternatively, as shown in FIG. 3b, the drug coating 10 may be applied to the balloon 2 in a folded configuration.

### Anti-Proliferative

In accordance with the present disclosure, the drug coating may further comprise an antiproliferative active ingredient. In general, the antiproliferative active ingredient may be any therapeutic agent capable of inhibiting the cellular hyperplasia response generally associated with balloon angioplasty. Example antiproliferative ingredients include the immunosuppressant sirolimus and its derivatives including everolimus, zotarolimus, biolimus, tacrolimus and pimecrolimus, to name a few. Other example antiproliferative agents include the chemotherapy agent paclitaxel and hydrates of paclitaxel. Example antiproliferative agents for the application of treating eye disease in the ICA and/or ophthalmic artery include sirolimus and paclitaxel.

The drug coating may comprise one or more drugs in a therapeutically effective amount. As used herein, “therapeutically effective amount” means a drug amount capable of inducing a therapeutic or preventive effect against the disease being treated or prevented. For example, if the disease being treated or prevented is restenosis of vascular tissue, the one or more drugs present in the drug coating may be present in an amount effective to treat or prevent restenosis of the treated vascular tissue in the patient. The drug coating may comprise one or more drugs in any suitable density. For example, the drug may be present in the drug coating at a density from about 0.1 μg/mm2 to about 100μg/mm2 , such as between about 0.25 μg/mm2 to about 20 μg/mm2.

In accordance with the current disclosure, the therapeutic agent may be in a purely amorphous state, a semi-crystalline state comprising a mixture of amorphous and crystalline regions, or in a purely crystalline state. In further accordance with the disclosure, the crystalline state may comprise any combination of known crystalline polymorphs of the therapeutic agent. Both the type and degree of crystallinity may be controlled through the coating process conditions. Alternatively, post-coating processes such as thermal annealing or solvent vapor exposure may be used to affect the crystalline state of the therapeutic agent subsequent to the coating process.

### Excipient

In addition to the therapeutic anti-proliferative active agent, the drug coating may include at least one additional ingredient commonly known as an excipient. The purpose of the excipient is to enable the delivery of clinically efficacious tissue levels of the active ingredient at the lesion site. This is accomplished by at least one several mechanisms. First, the excipient may increase the durability of the drug coating to reduce particulation and loss of drug during delivery of the balloon catheter to the lesion site. In addition, the excipient may increase the amount of drug that gets transferred to the lesion site upon expansion of the balloon. Finally, the excipient may extend duration of tissue levels for the active ingredient by modulation of the drug dissolution rate subsequent to tissue transfer.

In accordance with the present disclosure, the excipient may generally comprise a carotenoid compound. Carotenoids are photoactive pigments produced by plants, algae, bacteria, and fungi. Carotenoids are more generally known as tetraterpenoids. In general, carotenoids absorb wavelengths ranging from 400 to 550 nanometers (violet to green light). Carotenoids can be further categorized into two classes, xanthophylls and carotenes. (See, for example, Rao 2007.) In general, xanthophylls are oxygen containing versions of carotenoids. The presence of oxygen makes them more polar and water soluble than carotenes. U.S. Pat. No. 9,375,329B2 describes the use of a carotenoid as a biodegradable structural element of a stent. EP1362603B1 further describes the use of a carotenoid as a therapeutic agent stent coating. U.S. Pat. No. 8,241,249B2 also describes the use of carotenes as excipients within a balloon drug coating.

A general chemical structure of xanthophylls is shown in FIG. 3. In accordance with this embodiment, a xanthophyll is defined as any chemical compound according to FIG. 3 wherein at least one carbon of pendant group c1-6, or pendant group c1′-c6′ is also chemically bonded to oxygen. Preferred xanthophylls of the present invention include, but are not limited to, lutein, astaxanthin, zeaxanthin, meso zeaxanthin, cryptotaxanthin, tunaxanthin, salmoxanthin, and parasiloxanthin, to name a few. In general a given xanthophyll may exist in any stereoisomeric form.

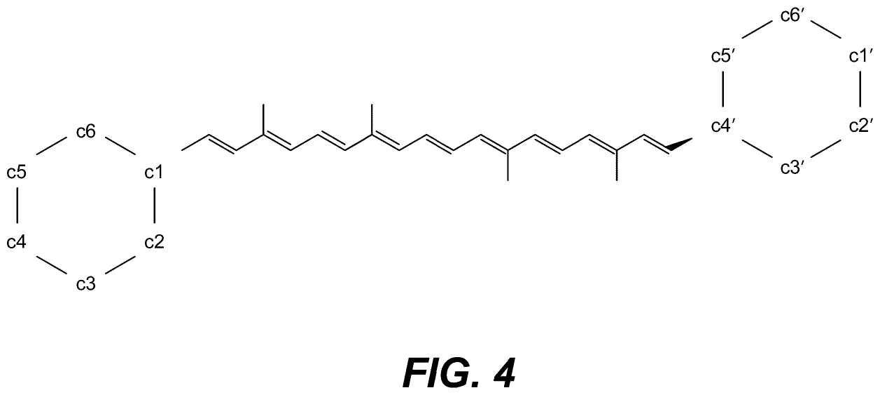

Example xanthophylls include lutein, meso-zeaxanthin, and zeaxanthin. In the eye, lutein, meso-zeaxanthin, and zeaxanthin are present as macular pigments of importance in visual function. (See, for example, Bernstein et. al., 2016.) The chemical structures of lutein, zeaxanthin, and meso-zeaxanthin are shown in FIG. 4.

The ratio of excipient to drug within the drug coating may be any value which allows for transfer of a clinically efficacious amount of therapeutic agent to tissue upon balloon expansion. The percentage of drug relative to excipient may range from 1% to 99%, 25% to 75%, or 33% to 66%, for example.

In accordance with the current disclosure, the therapeutic agent may be in a purely amorphous state, a semi-crystalline state comprising a mixture of amorphous and crystalline regions, or in a purely crystalline state. In further accordance with the disclosure, the crystalline state may comprise any combination of known crystalline polymorphs of the therapeutic agent. Both the type and degree of crystallinity may be controlled through the coating process parameters which are dependent on the process selected for the coating application as well as the therapeutic and surface-active agents. Each coating process lends itself to different coating morphologies and/or drug eluting properties. In the example of spray, typical process condition parameters include, viscosity of coating, distance of spray tip from balloon target, diameter of spray tip exit hole, pressure of air source delivering coating, path of motion of spray tip, pattern of layering of excipient then drug then excipient is determined, drying time between coatings and drying time prior to packaging. Alternatively, post-coating processes such as thermal annealing or solvent vapor exposure may be used to affect the crystalline state of the excipient subsequent to the coating process.

### Additional Ingredients

In further accordance with the present disclosure, the drug coating comprising an anti-proliferative and xanthophyll may further comprise any additional ingredient known to improve the functional performance of such coatings. Such additional ingredients may include, for example, surface active agents which improve the adhesion of the drug coating to either the balloon or tissue surface. These surface active agents may include (1) biodegradable polymers, such as methacrylate, acrylate, polyethylene glycol, or acrylate vinyl pyrrolidone; (2) biocompatible polymers, such as carbopol polymers, carbopol poloxamer gels, hydroxypropylmethylcellulose, guar gum, sodium carboxymethyl cellulose, polyvinyl pyrrolidone, hydroxypropyl cellulose, polycarbophil, polyacrylic acid, chitosan, starch, alginate, and their copolymers; (3) bioadhesive polymers, such as polylactic-co-glycolic acid (PLGA); and proteins and carbohydrates, such as gellan and gelatin, xanthan, mannitol, and chitosan. Further additional ingredients may include agents known to modulate the structure of the drug such as nucleating agents to affect particle size, agents which impact the degree of drug crystallinity which may also include plasticizers to reduce crystallinity. Other additional ingredients may include those which improve the stability of the drug coating such as, for example, anti-oxidants. Also included are any agents which further improve the pharmacokinetics of the therapeutic agent subsequent to tissue transfer. These may include, for example, anti-inflammatoirech as steroids and any agents which chemically interact with the therapeutic to optimize the pharmacokinetic properties of the delivered drug such as tissue levels over time. Additional ingredients may further comprise vascular growth factors such as the vascular endothelial growth factor VEGF-A and anti-vascular growth factors including anti-VEGF agents such as monoclonal antibodies such as bevacizumab, antibody derivatives such as ranibizumab, or orally-available small molecules that inhibit the tyrosine kinases stimulated by VEGF such as sunitinib, sorafenib, axitinib, and pazopanib.

### Delivery Mechanism/Particle Size

The present disclosure generally includes any procedure resulting in the transfer of a clinically efficacious dosage of the therapeutic agent to the desired location of treatment. For a balloon catheter, this may first include creation of access to the venous or arterial system of a patient. Access points may include, for example, the femoral artery, the radial artery, the carotid artery, jugular vein, subclavian vein, and femoral vein, to name a few. The distal end of the catheter may be tracked to the lesion site usually aided by an imaging modality such as x-ray fluoroscopy or magnetic resonance imaging. Tracking of the catheter may be facilitated with the use of a guide wire over which the lumen of the inner shaft passes. The guide wire may be inserted over the entire length of shaft from proximal to distal in an over the rail configuration, or the guide wire may pass through the inner lumen at the distal end of the catheter in a mono-rail configuration. The balloon may be inflated by any means capable of achieving a desired diameter in the expanded state. A method of inflation is with a syringe containing saline via the inflation lumen located between the inner and outer shaft. The balloon is generally inflated to a desired pressure corresponding to a balloon diameter determined by the compliance specifications of the balloon. The balloon may be maintained in the inflated state for any amount of time necessary to deliver the drug. A clinically efficacious amount of therapeutic agent may be transferred to the lesion site associated tissue by any number of mechanisms. For example, the drug may partition into the tissue by molecular dissolution from the balloon surface. Conditions for dissolution from a point of release to a target tissue rely on mechanisms of drug transport and binding such as diffusion, ion exchange, osmosis, convection and partitioning in and around the target tissue. These mechanisms are heavily dependent on the physicochemical properties of the drug and the surrounding environment. For example, hydrophobicity or absolute charge can regulate transport and distribution of a drug in and around arterial tissues. Alternatively, the drug coating including therapeutic agent and excipient may fragment into particulates which are embedded into the tissue upon balloon expansion. This may include any distribution of particle size resulting in a clinically efficacious amount of therapeutic agent. Exemplary ranges of particle size range from 5 μm to 50 μm. Furthermore, to create a clinically efficacious amount of therapeutic agent, the drug containing fragments may dissolve over a clinically relevant time period. This time period may range from minutes to months. Exemplary ranges include initiation of dissolution beginning at 1 hr after treatment and continuing up to 180 days. The drug amount at 180 days may still be in the therapeutic range and provide biological effect.

### EXAMPLES

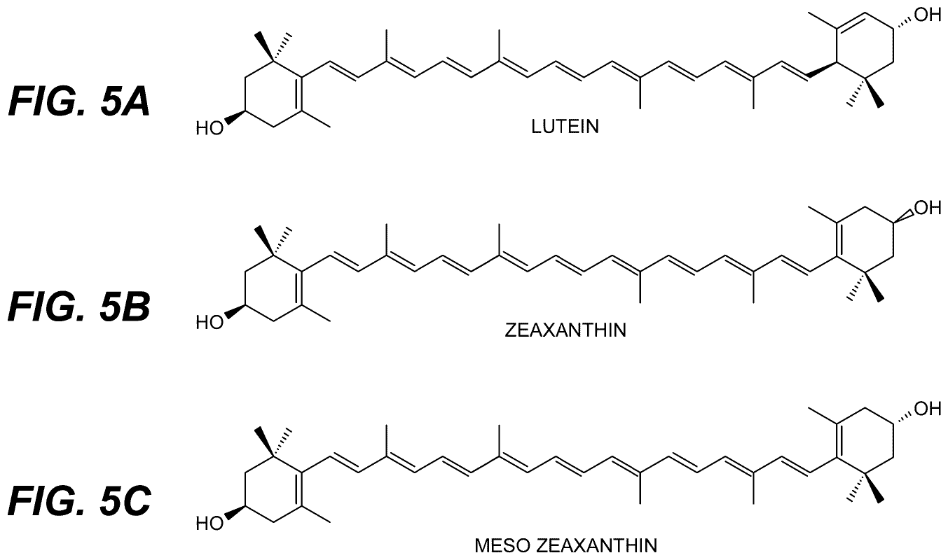

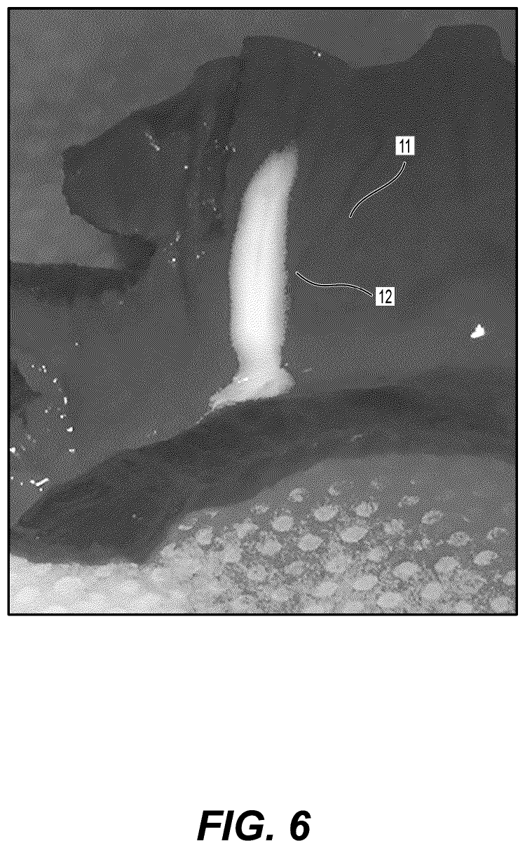

Example 1. A drug coating solution was prepared by dissolving 2.5 g of lutein (Sigma) and 2.5g sirolimus (AdooQ Bioscience) into 100 ml acetone (Sigma). In order to facilitate optical imaging of the drug coating a small amount (0.05 g) of fluorescein (Sigma) dye was also added to the solution. The drug coating solution was then coated onto a 6 mm length balloon catheter having a folded polyurethane balloon with nominal expanded diameter of 2 mm using a hand held, gas-assisted air brush sprayer. The drug coating was applied to a corresponding drug dose density of approximately 5 micrograms per square millimeter. As a substrate to demonstrate transfer of drug coating upon balloon expansion, a porcine heart was obtained and sectioned laterally across the left and right atrium to expose the cardiovascular arteries descending from the aorta. The balloon catheter of Example 1 was inserted into an accessible artery of appropriate diameter and expanded to a nominal diameter of 2 mm. Upon deflation and removal of the balloon catheter, the arterial region was imaged under ultraviolet light to detect transferred drug coating. FIG. 5 shows the optical image of the arterial region 11 with green colored (rendered white in image) areas 12 corresponding to regions fluorescing green from the added dye. Thus, demonstrating transfer of the drug coating to the arterial endothelial tissue upon expansion of the balloon. Current literature suggests that for currently commercially available drug coated balloons, <20% of the drug loaded onto the balloon is transferred to the vessel wall during inflation, with the remaining drug being shed as particulate into vessels downstream of the treatment site.
Example 2. In this example, a 12 mm long drug-coated polyurethane balloon with nominal inflated diameter of 1.25 mm was prepared as in Example 1, except the balloon was first inflated prior to coating. The drug coated balloon was then re-folded by deflation of the balloon. The deflated balloon was then inserted into an available artery and processed as in Example 1. FIG. 6 shows the subsequent fluorescent image of the exposed arterial region 12 demonstrating transfer of the drug coating.

1. An intravascular device including a transferable drug coating adhered to an exposed outer surface on the device, the drug coating comprising an anti-proliferative active ingredient and a xanthophyll excipient.

2. An intravascular device as in claim 1, wherein the xanthophyll is selected from the group consisting of lutein, astaxanthin, zeaxanthin, meso zeaxanthin, cryptotaxanthin, tunaxanthin, salmoxanthin, and parasiloxanthin, and mixtures thereof.

3. An intravascular device as in claim 1 wherein the anti-proliferative active ingredient is selected from the group consisting of sirolimus, everolimus, zotarolimus, bioliimus, tacrolimus, pimecrolimus, paclitaxel, and mixtures thereof.

4. An intravascular device as in claim 1, wherein the device comprises a balloon catheter having an expandable balloon, and wherein the drug coating is applied onto the balloon in an expanded configuration.

5. An intravascular device as in claim 1, wherein the device comprises a balloon catheter having an expandable balloon, and wherein the drug coating is applied onto the balloon in a folded configuration.

6. An intravascular device as in claim 1, further comprising an additional ingredient selected from the group consisting of adhesion agents, nucleating agents, stabilizing agents, anti-inflammatory agents, vascular growth factors and anti-vascular growth factors.

7. An intravascular device as in claim 1, wherein the device comprises a stent.

8. An intravascular device as in claim 1, wherein the device comprises a stent retriever.

9. An intravascular device as in claim 1, wherein the device comprises an aneurysm coil.

10. An intravascular device as in claim 1, wherein the device comprises a guidewire.

11. An intravascular device as in claim 1, wherein the device comprises an aspiration catheter.

12. An intravascular device as in claim 1, wherein the device comprises a microcatheter.

13. A method for treating a vascular stenosis comprising the steps of: providing an intravascular device with an expandable portion and a transferable drug coating disposed on an outer surface of the expandable portion, wherein the drug coating comprises an anti-proliferative active ingredient and a xanthophyll excipient; expanding the expandable portion such that the drug coating contacts the vascular stenosis; and transferring the drug coating from the expandable portion to the stenosis.

14. The method of claim 13 wherein the xanthophyll is selected from the group consisting of lutein, zeaxanthin, and mixtures thereof.

15. The method of claim 13 wherein the anti-proliferative active ingredient is selected from the group consisting of sirolimus, everolimus, zotarolimus, bioliimus, tacrolimus, pimecrolimus, paclitaxel, and mixtures thereof.

16. The method of claim 13 wherein the drug coating further comprises an additional ingredient selected from the group consisting of adhesion agents, nucleating agents, stabilizing agents, anti-inflammatory agents, vascular growth factors and anti-vascular growth factors.

17. A method for treating an eye disorder using an intravascular device with an expandable portion and a transferable drug coating disposed on an outer surface of the expandable portion, wherein the drug coating comprises an anti-proliferative active ingredient and a xanthophyll excipient, the method comprising: placing the expandable portion near a stenosis in an ophthalmic artery (OA), an internal carotid artery (ICA) or a junction between the OA and ICA; expanding the expandable portion such that the drug coating contacts the stenosis; and transferring the drug coating from the expandable portion to the stenosis.

18. The method of claim 17 wherein the xanthophyll is selected from the group consisting of lutein, zeaxanthin, and mixtures thereof.

19. The method of claim 17 wherein the anti-proliferative active ingredient is selected from the group consisting of sirolimus, everolimus, zotarolimus, bioliimus, tacrolimus, pimecrolimus, paclitaxel, and mixtures thereof.

20. The method of claim 17 wherein the drug coating further comprises an additional ingredient selected from the group consisting of adhesion agents, nucleating agents, stabilizing agents, anti-inflammatory agents, vascular growth factors and anti-vascular growth factors.
